SEQUENCE LISTING (4 sequences)

SEQ ID NO: 1 - DNA, 638 bp, synthetic construct
  misc_feature 1..638: note = Membrane-bound IL-15/CD8a fusion protein
  source 1..638: mol_type = other DNA
gaattcgcccttccaccatggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctaactcgag

SEQ ID NO: 2 - protein, 204 aa, synthetic construct
  REGION 1..204: note = Membrane-bound IL-15/CD8a fusion protein
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 3 - DNA, 489 bp, Homo sapiens
  source 1..489: mol_type = genomic DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 4 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

(19) United States
(12) Patent Application Publication
(10) Pub. No.: US 2025/0320457 A1
(43) Pub. Date: Oct. 16, 2025

(54) Natural Killer Cells Expressing Membrane-Bound Interleukin 15 (mbIL15) and Uses Thereof

(71) Applicants: National University of Singapore, Singapore (SG); St. Jude Children's Research Hospital, Inc., Memphis, TN (US)

(72) Inventors: Dario Campana, Singapore (SG); David Shook, Memphis, TN (US); Masaru Imamura, Chuo-ku (JP)

(21) Appl. No.: 19/054,108

(22) Filed: Feb. 14, 2025

Related U.S. Application Data
(63) Continuation of application No. 18/067,199, filed on Dec. 16, 2022, now Pat. No. 12,264,335.
Application No. 18/067,199 is a continuation of application No. 16/986,742, filed on Aug. 6, 2020, now Pat. No. 11,560,548.
Application No. 16/986,742 is a continuation of application No. 16/550,548, filed on Aug. 26, 2019, now Pat. No. 10,774,311.
Application No. 16/550,548 is a division of application No. 15/309,362, filed on Nov. 7, 2016, now Pat. No. 10,428,305.
(60) Provisional application No. 61/993,494, filed on May 15, 2014.

(51) Int. Cl. C12N 5/0783; A61K 38/20; A61K 40/15; A61K 40/42; C07K 14/54; C07K 14/705; C12N 5/00; C12N 15/62

(52) U.S. Cl. CPC C12N 5/0646; A61K 38/2013; A61K 38/2086; A61K 40/15; A61K 40/42; C07K 14/5443; C07K 14/70517; C12N 5/0006; C12N 15/62; A61K 2239/31; A61K 2239/38; A61K 2239/48; C07K 2319/03; C12N 2501/2315; C12N 2510/00

(57) ABSTRACT

The present invention provides, in certain aspects, a natural killer (NK) cell that expresses all or a functional portion of interleukin-15 (IL-15), and methods for producing such cells. The invention further provides methods of using a natural killer (NK) cell that expresses all or a functional portion of interleukin-15 (IL-15) to treat cancer in a subject or to enhance expansion and/or survival of NK cells.

### RELATED APPLICATIONS

This application is a continuation of U.S. application Ser. No. 18/067,199, filed Dec. 16, 2022, which is a continuation of U.S. application Ser. No. 16/986,742, filed Aug. 6, 2020, now U.S. Pat. No. 11,560,548, issued on Jan. 24, 2023, which is a continuation of U.S. application Ser. No. 16/550,548, filed Aug. 26, 2019, now U.S. Pat. No. 10,774,311, issued on Sep. 15, 2020, which is a divisional of U.S. application Ser. No. 15/309,362, filed Nov. 7, 2016, now U.S. Pat. No. 10,428,305, issued on Oct. 1, 2019, which is the U.S. National Stage of International Application No. PCT/SG2015/050111, filed on May 14, 2015, published in English, which claims the benefit of U.S. Provisional Application No. 61/993,494, filed on May 15, 2014. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 4459.1097-006 Sequence Listing.xml; created Feb. 14, 2025, 6,707 Bytes in size.

### BACKGROUND OF THE INVENTION

Survival and proliferation of NK cells in vivo requires stimulation by cytokines, such as IL-2 and IL-15. For example, after injection in immunodeficient mice, activated NK cells became undetectable after 1 week but persisted for up to one month if human IL-2 was also administered. Hence, clinical protocols using NK cell infusions typically rely on IL-2 administration to prolong NK cells survival in patients. However, IL-2 can have considerable side effects. In addition to fever and chills, IL-2 administration can lead to more serious and potentially fatal consequences, such as capillary leak syndrome. Decreasing the dose of IL-2 should reduce the risk of side effects but can result in stimulation of regulatory T cells which can inhibit NK cell function and possibly nullify its anti-cancer effect.

Hence, it would be important to develop alternative ways to promote NK cell expansion and activity in vitro and/or in vivo.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1C: Design and expression of IL-15 constructs. 1A. Schematic representation of the wild-type and membrane-bound IL-15 constructs (“wtIL15” and “mbIL15”) used in this study. 1B. Expression of IL-15 on the surface of NK cells transduced with mbIL15. Expanded NK cells were transduced with wtIL15, mbIL15 or with a vector containing GFP alone (“Mock”). Flow cytometry dot plots illustrate expression of GFP and IL-15, as detected by an anti-IL15 antibody (R&D Systems) and a goat-anti-mouse secondary antibody conjugated to phycoerythrin (Southern Biotechnology Associates). Percentage of cells (>98% CD56+CD3-NK cells) in each quadrant is shown. 1C. Secretion of IL-15 by NK cells transduced with wtIL15. NK cells from 3 different donors were tested in triplicate. Bars indicate mean±SD of ELISA measurements performed in supernatants collected after 24 and 48 hours of culture without IL-2. No IL-15 was detected in the supernatants of mock-transduced cells.

FIGS. 2A-2C: Survival and expansion of NK cells expressing IL-15 in vitro. 2A. Percentage of NK cell recovery as compared to input cells after 7-day parallel cultures without IL-2 for mock- and mbIL15 transduced cells from 15 donors (left panel) and mbIL15- or wtIL15-transduced cells from 9 donors (right panel). Horizontal bars indicate median value. Results of paired t tests are shown. Results of cultures with IL-2 (10 and 100 IU/mL) are shown in FIG. 6B. Survival and expansion of mock- and mbIL15-transduced NK cells from 6 donors with low dose IL-2 (10 IU/mL). 2C. Expansion and long-term survival of NK cells from one donor transduced with mbIL15, wtIL15 or mock-transduced cultured with no IL-2 or low dose IL-2 (results with 100 IU/mL IL2 are shown in FIG. 6). Percentage of NK cell recovery at the indicated days of culture is shown.

FIG. 3A-3C: Survival and expansion of NK cells expressing mb-IL15 in vivo. 3A. Absolute number of human CD45+ cells in peripheral blood of mice injected with mock- or mbIL15 transduced NK cells with or without IL-2 (16 mice total) 7 and 11 days after infusion (P=0.004 with no IL-2, P=0.021 with IL-2 on day 7; P=0.044 and 0.026 on day 11). 3B. Flow cytometric dot plots illustrate the presence of human CD45+, GFP+ NK cells in mouse peripheral blood without (top) and with IL-2 treatment (bottom). Percentages of human CD45+ cells with or without GFP expression is shown. 3C. Percentage of human CD45+ cells in various tissues of mice injected with mock- or mbIL15 transduced NK cells with or without IL-2 collected 11 days after injection. Collectively, percentages of human CD45+ cells were significantly higher with mbIL15 (P<0.001 with no IL-2, P=0.002 with IL-2).

FIGS. 4A-4C: Properties of NK cells expressing mbIL15. 4A. Relative proportion of GFP+ cells before and after 7 days of culture among NK cell populations transduced with mbIL15 or mock-transduced. Results with NK cells from 13 donors are shown; P<0.001 for mbIL15, not significant for mock. 4B. Immunophenotypic features of mbIL15-transduced NK cells. Cells marker analysis by flow cytometry was performed on NK cells cultured for 48 hours without IL-2. All results are summarized in the Table. 4C. Mock- and mbIL15-transduced NK cells were cultured for 48 hours without IL-2 and cell lysates were analyzed by Kinex Antibody Microarray (Kinexus). Of 809 anti-phosphoprotein antibodies tested, shown are those whose signals had a Z-ratio >0.5 and a % Error Range <100. Bars indicate percent signal change in NK cells expressing mbIL15 as compared to the normalized intensity in mock-transduced NK cells.

FIGS. 5A-5D: Anti-tumor capacity of NK cells expressing mbIL15. 5A. Results of 24-hour cytotoxicity assays with mbIL15- and mock-transduced NK cells from 9 donors against the Nalm-6, U937, K562, Daudi, SK-BR-3, and ES8 cell lines at 1:4 and 1:1 E:T ratio (15 experiments at each ratio; P<0.001 for both). Results obtained with individual cell lines in 4-hour and 24-hour cytotoxicity assays are shown in FIG. 7. 5B. NK cells expressing mbIL15 have an increased release of lytic granules in the presence of target cells. Percentage of CD107a+ NK cells after 4-hour cytotoxicity assays at 1:1 E: T. Results with NK cells from 3 donors against 2 cell lines are shown (P=0.007). 5C. NK cells expressing mbIL15 exert anti-tumor activity in vivo. NOD-SCID-IL2RGnull mice were injected i.p. with 1×10⁴ U937 cells labeled with luciferase. In 3 mice, no treatment was given (“No NK”), while 4 mice received mock-transduced NK cells (1×10⁷ i.p.) on days 3 and 7, and 4 other mice mbIL15-transduced NK cells at the same dose and schedule. Results of in vivo imaging of tumor growth are shown (ventral images). 5D. Overall survival comparisons of mice in the different treatment groups. Mice were euthanized when bioluminescence reached 1×10¹¹ photons/second. P values for log rank test of the 3 curves, and for comparisons between each of 2 curves are shown.

FIGS. 6A-6C: Survival and expansion of NK cells expressing IL-15 in vitro. 6A. Expansion of NK cells expressing mbIL15 in the absence of IL-2 is suppressed by an anti-IL-15 neutralizing antibody. Symbols show mean (±SD; n=3) NK cell recovery during culture as compared to input cells in experiments with NK cells transduced with mbIL15. 6B. Percentage of NK cell recovery as compared to input cells after 7-day parallel cultures with low-(10 IU/mL) and high-dose (100 IU/mL) IL-2 for mock-, mbIL15- and wtIL15-transduced cells from 6 donors. Horizontal bars indicate median value. Results of paired t tests are shown. 6C. Expansion and long-term survival of NK cells from one donor transduced with mbIL15 or wtIL15 and cultured with 100 IU/mL IL2. Percentage of NK cell recovery at the indicated days of culture is shown.

FIGS. 7A-7B: Anti-tumor capacity of NK cells expressing mbIL15. Results of 4-hour (7A) and 24-hour cytotoxicity assays (7B) with mbIL15- and mock-transduced NK cells against the Nalm-6, U937, K562, Daudi, SK-BR-3, and ES8 cell lines at 1:4, 1:2 and 1:1 E:T ratio are shown. Each symbol indicates mean±SD cytotoxicity in experiments with NK cells from 3 different donors for U937, K562, ES8, and 2 donors for Nalm-6, Daudi and SK-BR-3, all performed in triplicate (P<0.001 for all experiments).

FIGS. 8A-8C: Anti-tumor capacity of NK cells expressing mbIL15. NOD-SCID-IL2RGnull mice were injected i.p. with 1×10⁵ ES8 cells labeled with luciferase. In 7 mice, no treatment was given (“No NK”), while 11 mice received mock-transduced NK cells (1×10⁷ i.p.) on day 3, and 12 other mice mbIL15-transduced NK cells at the same dose and schedule. 8A. Results of in vivo imaging of tumor growth. Ventral images of the 4 mice with the highest tumor signal in each group are shown. 8B. Results of in vivo imaging of tumor growth. Each symbol corresponds to one bioluminescence measurement (photon/second relative day 3 measurements in each mouse). 8C. Overall survival comparisons of mice in the different treatment groups. Mice were euthanized when bioluminescence reached 1×10¹⁰ photons/second. P values for log rank test of the 3 curves, and for comparisons between each of 2 curves are shown.

FIG. 9 shows the nucleotide sequence (SEQ ID NO: 1) and the amino acid sequence (SEQ ID NO: 2) of membrane bound IL-15.

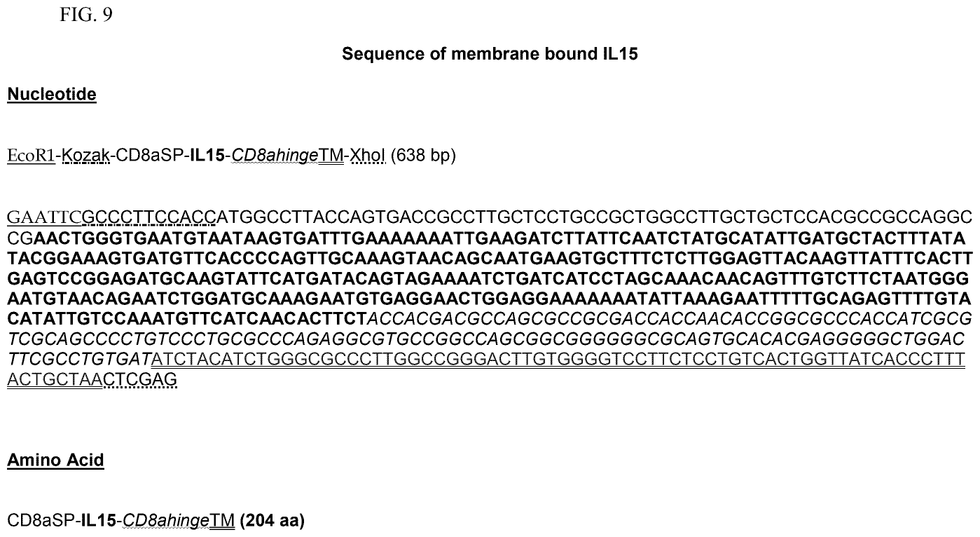

FIG. 10 shows the nucleotide sequence (SEQ ID NO: 3) and amino acid sequence (SEQ ID NO: 4) of human IL-15 (NCBI Reference Sequence: NM_000585.4).

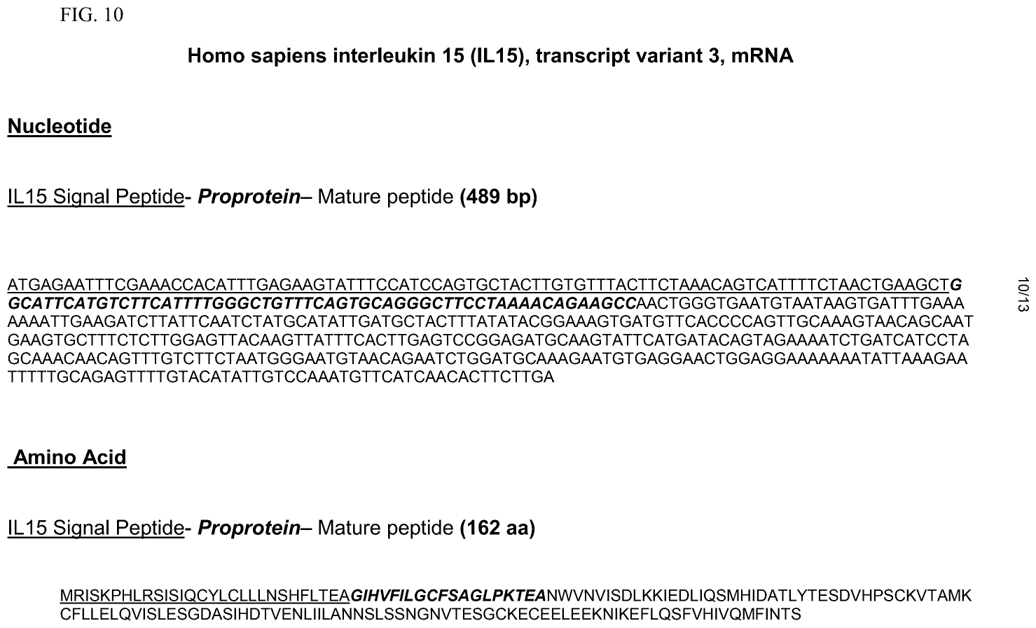

FIGS. 11A-11C: mbIL15 stimulates NK cells by cis presentation. 11A. NK92 cells were transduced with mbIL15 (left) or wtIL15 (right) in a vector containing GFP, sorted to obtain 100% GFP+ cells and co-cultured with untransduced NK92 cells at a 1:1 ratio. Shown is percentage of cell recovery (±SD; n=3) after culture for GFP+ and GFP-cells, relative to the number of cells at the beginning of the culture. 11B. NK92 cells expressing mbIL15 or untransduced were co-cultured with K562 cells (“K”) either transduced with mbIL15 or untransduced at 1:2 ratio in the combinations shown. K562 cells were labeled with PKH26 (Sigma) and treated with Streck cell preservative (Streck, Omaha, NE) to prevent cell division before culture. Shown is percentage of NK92 cell recovery (±SD; n=3) after culture, relative to cell numbers at the beginning of the culture. 11C. Proliferation of NK92 cells expressing mbIL15 compared to that of untransduced NK92 cells in the presence of increasing concentrations of exogenous IL-15. Cultures were performed in the absence of IL-2 (left), or with IL-2 at 10 IU/mL (center) or 100 IU/mL (right). Shown is percentage of cell recovery (±SD; n=3) after culture relative to the number of cells at the beginning of the culture.

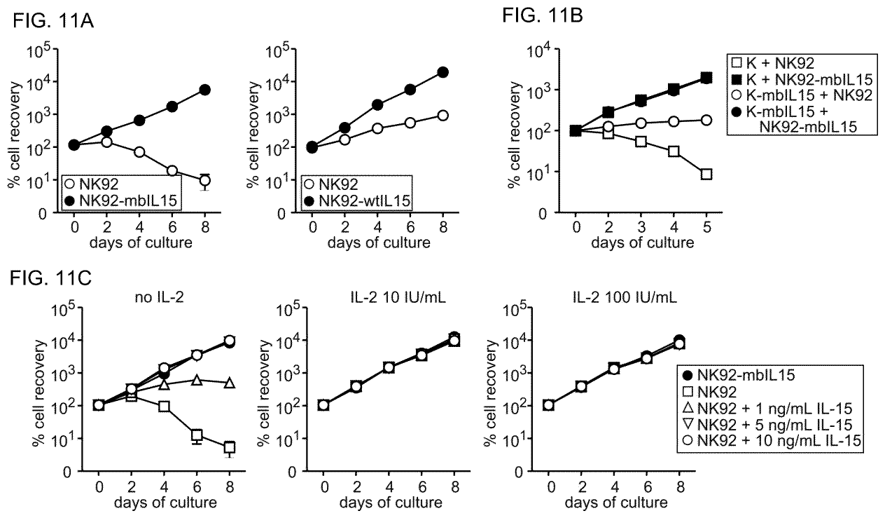

FIGS. 12A-12C. Expression and function of KIRs in mb15-NK cells. 12A. NK cell subsets defined by their KIR expression before transduction, and after mock- or mb15-transduction. Flow cytometric dot plots show results of staining with anti-KIR antibodies in CD56+CD3− cells from 2 donors. Percentages of KIR+ cells are shown. 12B. Results of CD107a expression in CD158a-positive and CD158a-negative subsets after 4-hour culture with 721.221 cells or the same cells expressing the CD158a-binding Cw6 HLA. Shown are mean (+SD) of 4 independent experiments with NK cells from 3 donors (** P<0.0001; * P=0.0002). 12C. Results of IFNγ secretion in the same experiments shown in 12B (** P<0.0001).

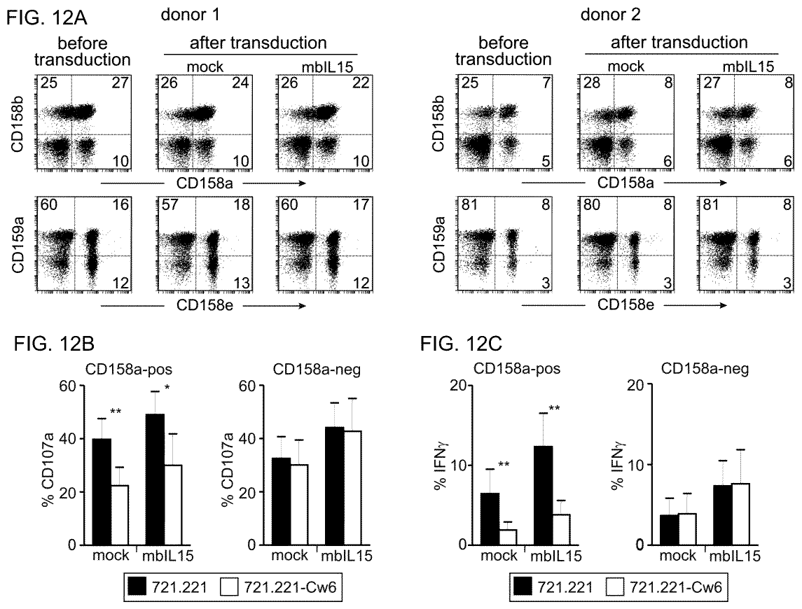

FIGS. 13A and 13B: Antibody-dependent cell cytotoxicity (ADCC) of NK cells expressing mbIL15. Results of 4-hour ADCC assays with mbIL15- and mock-transduced NK cells against (13A) Daudi and (13B) SK-BR-3 in the presence of Rituximab or Trastuzumab, respectively; IgG at the same concentration of the immunotherapeutic antibodies (1 μg/mL) was used as a control. Each symbol indicates mean±SD cytotoxicity in experiments with NK cells from each donor in triplicate. In the presence of immunotherapeutic antibodies, mbIL15-NK cells exerted significantly higher ADCC than mock-transduced cells (P<0.001 for either donor in tests with Daudi or SK-BR-3). Cytotoxicity by mbIL15-NK cells without antibody was also significantly higher (P<0.001 for either donor in tests with Daudi or SK-BR-3).

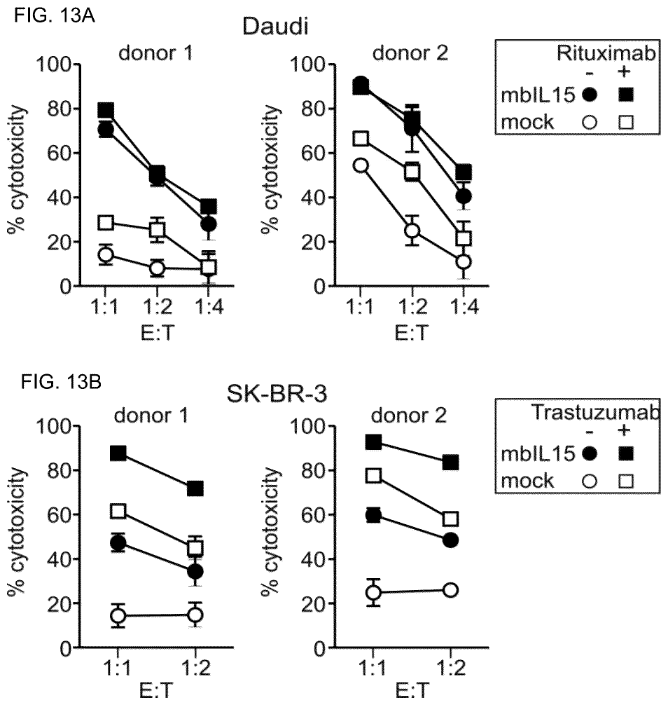

### DETAILED DESCRIPTION OF THE INVENTION

A description of example embodiments of the invention follows.

The well-established anti-leukemic activity of natural killer (NK) cells indicates therapeutic potential for NK cell infusions. NK cell survival and, hence, cytotoxicity requires cytokine support. Described herein are experiments investigating whether expression of interleukin-15 (IL-15) in a non-secretory, membrane-bound form could sustain NK cell growth. The human IL15 gene was linked to that encoding CD8α transmembrane domain (“mbIL15”). After retroviral transduction, human NK cells expressed mbIL-15 on the cell surface but IL-15 secretion was negligible. Survival and expansion of mbIL15-NK cells without IL-2 was vastly superior to that of mock-transduced cells (after 7-day culture, P<0.0001, n=15), and to that of NK cells secreting non-membrane bound IL-15 (P=0.025, n=9); viable mbIL15-NK cells were detectable for up to 2 months. In immunodeficient mice, mbIL15-NK cells expanded without IL-2, and were detectable in all tissues examined (except brain) in much higher numbers than mock-transduced NK cells (P<0.001). Expansion in vitro and in vivo further increased with IL-2. The primary mechanism of mbIL15 stimulation was autocrine; it activated IL-15 signaling and anti-apoptotic signaling. Cytotoxicity against leukemia, lymphoma and solid tumor cell lines was consistently higher with mbIL15-NK cells. Median 24-hour cytotoxicity at 1:4 E: T was 71% versus 22% with mock-transduced cells; at 1:1 E: T, it was 99% versus 54% (P<0.0001). Increased anti-tumor capacity was also evident in immunodeficient mice engrafted with leukemia (U937) or sarcoma (ES8) cells. Thus, mbIL15 conferred independent growth to NK cells and enhanced their anti-tumor capacity. Infusion of mbIL15-NK cells allows NK cell therapy without the adverse effects of IL-2.

Accordingly, provided herein is a (one or more; a plurality) cell that expresses all or a functional portion of interleukin-15 (IL-15), wherein the cell is a cell that responds to IL-15. A cell that responds to IL-15 includes a cell in which one or more of its activities are regulated by IL-15. Examples of such cells include natural killer (NK) cells, T-cells, dendritic cells and moncytes. The one or more (e.g., isolated) cells can express all or a functional portion of IL-15 as a membrane-bound polypeptide, as a secretory protein or as a combination thereof.

In one aspect, the invention is directed to a natural killer (NK) cell(s) that expresses all or a functional portion of interleukin-15 (IL-15). The one or more (e.g., isolated) NK cells can express all or a functional portion of IL-15 as a membrane-bound polypeptide, as a secretory protein or as a combination thereof.

As used herein, “Natural Killer Cells” (“NK cells”) refer to a type of cytotoxic lymphocyte of the immune system. NK cells provide rapid responses to virally infected cells and respond to transformed cells. Typically immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill target. NK cells are large granular lymphocytes (LGL) and are known to differentiate and mature in the bone marrow from where they then enter into the circulation.

In some aspects, the NK cell is a mammalian NK cell. Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. In a particular aspect, the mammalian NK cell is a human NK cell.

As used herein “Interleukin-15” (“IL-15”) refers to a cytokine that regulates T and NK cell activation and proliferation. This cytokine and interleukin 2 share many biological activities. They are found to bind common receptor subunits, and may compete for the same receptor, and thus negatively regulate each other's activity. The number of CD8+ memory cells is shown to be controlled by a balance between IL-15 and IL-2. This cytokine induces the activation of JAK kinases, as well as the phosphorylation and activation of transcription activators STAT3, STAT5, and STAT6 and may increase the expression of apoptosis inhibitor BCL2L1/BCL-x(L), possibly through the transcription activation activity of STAT6, and thus prevent apoptosis.

A “functional portion” (“biologically active portion”) of IL-15 refers to a portion of IL-15 that retains one or more functions of full length or mature IL-15. Such functions include the promotion of NK cell survival, regulation of NK cell and T cell activation and proliferation as well as the support of NK cell development from hematopoietic stem cells.

As will be appreciated by those of skill in the art, the sequence of a variety of IL-15 molecules are known in the art. In one aspect, the IL-15 is a wild type IL-15. In some aspects, the IL-15 is a mammalian IL-15 (e.g., Homo sapiens interleukin 15 (IL15), transcript variant 3, mRNA, NCBI Reference Sequence: NM_000585.4; Canis lupus familiaris interleukin 15 (IL15), mRNA, NCBI Reference Sequence: NM_001197188.1; Felis catus interleukin 15 (IL15), mRNA, NCBI Reference Sequence: NM_001009207.1). Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. In a particular aspect, the mammalian IL-15 is a human IL-15.

All or a functional portion of IL-15 can be expressed by one or more NK cells (as a membrane-bound and/or secreted polypeptide) in a variety of ways. For example, all or a functional portion of the IL-15 can be expressed within the NK cell and secreted from the NK cell and/or can be linked (conjugated; fused) directly or indirectly (e.g., ionic, non-ionic, covalent linkage) to the surface (e.g., at the surface, or within the membrane, of an NK cell) of the NK cell using any of a variety of linkers known in the art (Hermanson, G., Bioconjugate Techniques, Academic Press 1996). In particular aspects, all or a functional portion of the IL-15 is linked to all or a portion of a transmembrane protein. In one aspect, the NK cell expresses a fusion protein comprising all or a portion of IL-15 fused to all or a portion of a transmembrane protein. In a particular aspect, the portion of the transmembrane protein comprises all or a portion of a transmembrane domain of the transmembrane protein.

As used herein, a “transmembrane protein” or “membrane protein” is a protein located at and/or within a membrane such as the phospholipid bilayer of a biological membrane (e.g., biomembranes such as the membrane of a cell). Membrane proteins enable the membrane to carry out its distinctive activities. The complement of proteins attached to a membrane varies depending on cell type and subcellular location. Some proteins are bound only to the membrane surface, whereas others have one or more regions buried within the membrane and/or domains on one or both sides of the membrane. Protein domains on the extracellular membrane surface are generally involved in cell-cell signaling or interactions. Domains lying along the cytosolic face of the membrane have a wide range of functions, from anchoring cytoskeletal proteins to the membrane to triggering intracellular signaling pathways. Domains within the membrane, referred to herein as “transmembrane domains”, particularly those that form channels and pores, move molecules across the membrane. A “transmembrane domain”, is a three-dimensional protein structure which is thermodynamically stable in a membrane (e.g., a membrane of a vesicle such as a cell). Examples of transmembrane domains include a single alpha helix, a stable complex of several transmembrane alpha helices, a transmembrane beta barrel, a beta-helix of gramicidin A, or any other structure. Transmembrane helices are usually about 20 amino acids in length.

Typically, membrane proteins are classified into two broad categories—integral (intrinsic) and peripheral (extrinsic)—based on the nature of the membrane-protein interactions. Most biomembranes contain both types of membrane proteins.

Integral membrane proteins, also called intrinsic proteins, have one or more segments that are embedded in the phospholipid bilayer. Integral membrane proteins include transmembrane proteins and lipid-anchored proteins. Most integral proteins contain residues with hydrophobic side chains that interact with fatty acyl groups of the membrane phospholipids, thus anchoring the protein to the membrane. Most integral proteins span the entire phospholipid bilayer. These transmembrane proteins contain one or more membrane-spanning domains as well as domains, from four to several hundred residues long, extending into the aqueous medium on each side of the bilayer. Typically, the membrane-spanning domains are one or more (e.g., about 1, 2, 3, 4, 5, 6, 7, 8, 9, 10 or more) a helices and/or β strands. Membrane-spanning α-helical domains are typically embedded in membranes by hydrophobic interactions with the lipid interior of the bilayer and probably also by ionic interactions with the polar head groups of the phospholipids (e.g., glycophorin). The structure of β strands are typically in the form of membrane spanning barrels (e.g., porin). Some integral proteins are anchored to one of the membrane leaflets by covalently bound fatty acids. In these proteins, the bound fatty acid is embedded in the membrane, but the polypeptide chain does not enter the phospholipid bilayer. Some cell-surface proteins are anchored to the exoplasmic face of the plasma membrane by a complex glycosylated phospholipid that is linked to the C-terminus (e.g., glycosylphosphatidylinositol, alkaline phosphatase). Some cytosolic proteins are anchored to the cytosolic face of membranes by a hydrocarbon moiety covalently attached to a cysteine near the C-terminus (e.g., prenyl, farnesyl, and geranylgeranyl groups). In another group of lipid-anchored cytosolic proteins, a fatty acyl group (e.g., myristate or palmitate) is linked by an amide bond to the N-terminal glycine residue.

Peripheral membrane proteins, or extrinsic proteins, do not interact with the hydrophobic core of the phospholipid bilayer. Instead they are usually bound to the membrane indirectly by interactions with integral membrane proteins or directly by interactions with lipid polar head groups. Peripheral proteins localized to the cytosolic face of the plasma membrane include the cytoskeletal proteins spectrin and actin in erythrocytes and the enzyme protein kinase C. This enzyme shuttles between the cytosol and the cytosolic face of the plasma membrane and plays a role in signal transduction. Other peripheral proteins, including certain proteins of the extracellular matrix, are localized to the outer (exoplasmic) surface of the plasma membrane.

Examples of transmembrane proteins include a receptor, a ligand, an immunoglobulin, a glycophorin or a combination thereof. Specific examples of transmembrane proteins include CD8α, CD4, CD3ε, CD3γ, CD3δ, CD3ζ, CD28, CD137, FcεRIγ, a T-cell receptor (TCR such as TCRα and/or TCRβ), a nicotinic acetylcholine receptor, a GABA receptor, or a combination thereof. Specific examples of immunoglobulins include IgG, IgA, IgM, IgE, IgD or a combination thereof. Specific examples of glycophorin include glycophorin A, glycophorin D or a combination thereof.

In addition to being linked to all or a portion of a transmembrane protein, all or a functional portion of the IL-15 can be linked to other components such as a signal peptide (e.g., a CD8α signal sequence), a leader sequence, a secretory signal, a label (e.g., a reporter gene), etc. In a particular aspect, the all or a functional portion of IL-15 is fused to a signal peptide of CD8α and all or a portion of a transmembrane domain of CD8α.

In another aspect, the invention is directed to a method of producing a natural killer (NK) cell that expresses all or a functional portion of interleukin-15 (IL-15). All or a portion of the IL-15 can be expressed as a membrane-bound polypeptide, a secreted polypeptide or as a combination thereof. The method comprises introducing nucleic acid encoding all or a functional portion of IL-15 into the one or more NK cells. In one aspect, the nucleic acid encoding all or a functional portion of IL-15 is linked (e.g., fused) to all or a portion of a transmembrane protein. Alternatively, or in addition, nucleic acid encoding all or a functional portion of IL-15 is introduced into the NK cell (e.g., wild type IL-15). As will be apparent to those of skill in the art, aspects in which nucleic acid encoding all or a functional portion if IL-15 and all or a functional portion of IL-15 fused to all or a portion of a transmembrane protein is introduced in to NK cell, can be done so using a single nucleic acid or multiple (e.g., separate; two) nucleic acids. The NK cell is maintained under conditions in which all or a functional portion of the IL-15 is expressed as a membrane-bound polypeptide and/or as a secreted polypeptide thereby producing a NK cell that expresses all or a functional portion of IL-15 as a membrane-bound polypeptide and/or as a secreted polypeptide. In a particular aspect, nucleic acid encoding all or a functional portion of IL-15 is fused to a signal peptide of CD8α and all or a portion of a transmembrane domain of CD8α is introduced into the NK cell.

In yet another aspect, the invention is directed to a method of enhancing expansion and/or survival of NK cells (e.g., in vitro, ex vivo, and/or in vivo). The method comprises introducing nucleic acid encoding all or a functional portion of IL-15. Nucleic acid encoding all or a portion of the IL-15 (e.g., wild type IL-15) and/or encoding all or a functional portion of IL-15 fused to all or a portion of a transmembrane protein can be introduced into the NK cell. Thus, the NK cell can express all or a functional portion of IL-15 as a membrane-bound polypeptide, a secreted polypeptide or as a combination thereof. The NK cells are maintained under conditions in which all or a portion of the IL-15 is expressed as a membrane-bound polypeptide, a secreted polypeptide or as a combination thereof and in which the NK cells proliferate. In a particular aspect, nucleic acid encoding all or a functional portion of IL-15 is fused to a signal peptide of CD8α and all or a portion of a transmembrane domain of CD8α is introduced into the NK cell. In some aspects, the method can further comprise contacting the NK cells comprising membrane-bound IL-15 and/or secreted IL-15 with IL-2. In some aspects, the concentration of IL-2 is from about 10 IU/ml to about 1000 IU/ml. In other aspects, the concentration of IL-2 is about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720 740, 760, 780, 800, 820, 840, 860, 880, 900, 920, 940, 960, 980 IU/ml.

As will be apparent to those of skill in the art, a variety of methods for introducing nucleic acid (e.g., transfection, transduction, and/or transposon system) encoding all or a functional portion of IL-15 as a transmembrane polypeptide and/or as a secreted polypeptide into a NK cell can be used. Examples of such methods include chemical-based methods (e.g., involving the use of calcium phosphate; highly branched organic compounds (e.g., dendrimers); liposomes (lipofection); and/or cationic polymers (e.g., DEAE dextran; polyethylenimine)), non-chemical-based methods (e.g., electroporation; cell squeezing; sonoporation; optical transfection; impalefection; hydrodynamic delivery), particle-based methods (e.g., gene gun; magnetofection; particle bombardment), vector-based methods (e.g., vectors including viral vectors such as retroviral vector, lentiviral vectors, adenoviral vectors, etc.), nucleotransfection, transposon-based methods (e.g., Sleeping Beauty, PiggyBAC, etc.) and/or RNA transfection.

Also apparent to those of skill in the art is that a variety of methods of maintaining NK cells under conditions in which (i) all or a functional portion of the IL-15 is expressed as a membrane-bound polypeptide and/or as a secreted polypeptide and/or (ii) the NK cells comprising membrane-bound IL-15 and/or secreted IL-15 proliferate can be used. For example, NK cells can be grown and/or maintained at an appropriate temperature and gas mixture (e.g., about 25° C. to about 37° C., about 5% CO₂ in a cell incubator). Culture conditions can vary widely, and variation of conditions for a particular cell type can result in different phenotypes. In addition to temperature and gas mixture, a commonly varied factor in culture systems is the cell growth medium. Recipes for growth media can vary in pH, glucose concentration, growth factors, and the presence of other nutrients. The growth factors used to supplement media are often derived from the serum of animal blood, such as fetal bovine serum (FBS), bovine calf serum, equine serum, porcine serum and/or human platelet lysate (hPL). Other factors considered for maintaining cells include plating density (number of cells per volume of culture medium) and growth of the cells in suspension or adherent cultures.

The methods can further comprise isolating or separating the one or more NK cells produced by the methods provided herein. In addition, the methods can further comprise culturing the one or more NK cells. In some aspects, an NK cell line is produced.

The invention also encompasses a (one or more) natural killer (NK) cell or cell line produced by the methods described herein, and compositions comprising the NK cells provided herein. In a particular aspect, the composition is a pharmaceutical composition comprising one or more of the NK cells or cell lines provided herein. The pharmaceutical composition can further comprise all or a functional portion of IL-2 (e.g., all or a functional portion of an (one or more) IL-2 protein; nucleic acid encoding all or a functional portion of IL-2).

As used herein, “IL-2” refers to a member of a cytokine family that also includes IL-4, IL-7, IL-9, IL-15 and IL-21. IL-2 signals through a receptor complex consisting of three chains, termed alpha, beta and gamma. The gamma chain is shared by all members of this family of cytokine receptors. IL-2, which similar to IL-15, facilitates production of immunoglobulins made by B cells and induces the differentiation and proliferation of NK cells. Primary differences between IL-2 and IL-15 are found in adaptive immune responses. For example, IL-2 is necessary for adaptive immunity to foreign pathogens, as it is the basis for the development of immunological memory. On the other hand, IL-15 is necessary for maintaining highly specific T cell responses by supporting the survival of CD8 memory T cells.

In another aspect, the invention is directed to a method of treating a disease and/or condition involving NK cell therapy in an individual in need thereof comprising administering to the individual a natural killer (NK) cell that expresses all or a functional portion of interleukin-15 (IL-15). In particular aspects, the NK cells express all or a functional portion of IL-15 as a membrane-bound polypeptide and/or as a secreted polypeptide. As is known in the art, diseases and/or conditions that involve NK cell therapy include NK cell deficiencies, cancer, autoimmune diseases, infectious diseases and the like.

In a particular aspect, the invention is directed to a method of treating cancer (e.g., a tumor) in an individual in need thereof comprising administering to the individual a natural killer (NK) cell that expresses all or a functional portion of interleukin-15 (IL-15). All or a functional portion of IL-15 can be expressed as a membrane-bound polypeptide and/or as a secreted polypeptide.

The method can further comprise administering one or more antibodies, antigenic fragments and/or fusions thereof specific to the cancer (e.g., tumor). For example, the method can further comprise administering one or more antibodies directed against one or more tumor antigens. As will be appreciated by those of skill in the art, the one or more antibodies can be a polyclonal antibody, a monoclonal antibody, a multivalent (e.g., bivalent, trivalent) antibody, a chimeric antibody, a humanized antibody, etc. and combinations thereof. A variety of antigenic fragments and/or fusions are also known in the art and include Fab′, F(ab′)₂, single chain variable fragment (scFv), multivalent scFv (e.g., di-scFv, tri-scFv), single domain antibody (nanobody) and etc.

In some aspects, the cancer is a leukemia (e.g., acute lymphoblastic leukemia; acute myeloid leukemia; chronic myelogenous leukemia, chronic lymphocytic leukemia), a myelodysplastic syndrome, a lymphoma (e.g., B cell non-Hodgkin lymphoma, Hodgkin lymphoma, T-cell lymphoblastic lymphoma, anaplastic large cell lymphoma), a solid tumor (e.g., a breast cancer, prostate cancer, gastric cancer, colon cancer, hepatocellular carcinoma, nasopharyngeal carcinoma, neuroblastoma, high grade glioma), a sarcoma (e.g., Ewing sarcoma, rhabdomyosarcoma, non-rhabdomyosarcoma soft-tissue sarcoma, osteosarcoma).

The method of treating cancer can further comprise administering IL-2 (all or a functional portion of IL-2 protein; nucleic acid encoding all or a functional portion of IL-2) to the individual. In one aspect, the IL-2 is mammalian IL-2, such as human IL-2. In a particular aspect, a low dose of the IL-2 is administered to the individual. As used herein, a “low dose” of IL-12 refers to a dose of IL-2 of about 1 million IU/m² or less (e.g., about 800,000 IU/m²; 600,000 IU/m²; 400,000 IU/m²; 200,000 IU/m²; 100,000 IU/m²; 80,000 IU/m²; 60,000 IU/m²; 40,000 IU/m²; 20,000 IU/m²; 10,000 IU/m²; 8,000 IU/m²; 6,000 IU/m²; 4,000 IU/m²; 2,000 IU/m²; 1,000 IU/m²; 800 IU/m²; 600 IU/m²; 400 IU/m²; 200 IU/m²; 100 IU/m²). In contrast, a normal dose of IL-2 is about 1 million IU/m² to about 5 million IU/m².

The one or more natural killer (NK) cell(s) that express all or a functional portion of interleukin-15 (IL-15) (e.g., therapeutic compound; pharmaceutical composition) are administered in a therapeutically effective amount (i.e., an amount that is sufficient to treat the cancer, such as by ameliorating symptoms associated with the cancer, preventing or delaying the onset of the cancer, also lessening the severity or frequency of symptoms of the cancer and/or preventing, delaying or overcoming metastasis of the cancer). The amount that will be therapeutically effective in the treatment of a particular individual will depend on the symptoms and severity of the condition (e.g., cancer), and can be determined by standard clinical techniques. In addition, in vitro or in vivo assays may optionally be employed to help identify optimal dosage ranges. The precise dose to be employed in the formulation will also depend on the route of administration, and the seriousness of the cancer, and should be decided according to the judgment of a practitioner and each patient's circumstances. Effective doses may be extrapolated from dose-response curves derived from in vitro or animal model test systems.

The therapeutic compound can be delivered in a composition (e.g., a pharmaceutical composition), as described above, or by themselves. They can be administered systemically, or can be targeted to a particular tissue. The therapeutic compounds can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. A combination of any of the above methods of treatment can also be used.

The compounds for use in the methods described herein can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc.

Compounds described herein can be formulated as neutral or salt forms.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In yet other aspects, the invention is directed to pharmaceutical compositions comprising one or more NK cells that expresses all or a functional portion of interleukin-15 (IL-15) as a membrane-bound polypeptide. The invention is also directed to compositions (e.g., pharmaceutical compositions) for use as a medicament in therapy. For example, the agents identified herein can be used in the treatment of cancer. In addition, the agents identified herein can be used in the manufacture of a medicament for the treatment of cancer.

As used herein an “individual” refers to an animal, and in a particular aspect, a mammal. Examples of mammals include primates, a canine, a feline, a rodent, and the like. Specific examples include humans, dogs, cats, horses, cows, sheep, goats, rabbits, guinea pigs, rats and mice. The term “individual in need thereof” refers to an individual who is in need of treatment or prophylaxis as determined by a researcher, veterinarian, medical doctor or other clinician. In one embodiment, an individual in need thereof is a mammal, such as a human.

An (one or more) “isolated,” “substantially pure,” or “substantially pure and isolated” NK cell, as used herein, is one that is separated from (substantially isolated with respect to) the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system, or reagent mix. In other circumstances, the material may be purified to essential homogeneity, for example, as determined by agarose gel electrophoresis or column chromatography such as HPLC. Preferably, an NK cell comprises at least about 50%, 80%, 90%, 95%, 98% or 99% (on a molar basis) of all macromolecular species present.

Articles such as “a”, “an”, “the” and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

### Exemplification

### Material and Methods

### Tumor Cell Lines

The human cell lines Nalm-6 (B-lineage acute lymphoblastic leukemia), Daudi (B-cell lymphoma), K562 and U937 (acute myeloid leukemia), and SK-BR-3 (breast carcinoma) were obtained from the American Type Culture Collection, the Ewing sarcoma cell line ES8 was from the St. Jude Children's Research Hospital tissue repository. All of the cell lines were transduced with a MSCV-internal ribosome entry site (IRES)-GFP retroviral vector (from the St. Jude Vector Development and Production Shared Resource) containing the firefly luciferase gene. Transduced cells were selected for their expression of GFP with a MoFlo (Beckman Coulter, Miami, FL) or a FACSAria (BD Biosciences, San Jose, CA). RPMI-1640 (Invitrogen, Carlsbad, CA) supplemented with 10% fetal bovine serum (FBS; Thermo Fisher Scientific, Waltham, MA) and antibiotics were used to maintain all cell lines. Cell lines were characterized by the providers for molecular and/or gene expression features; the cell marker profile of leukemia and lymphoma cell lines was periodically tested by flow cytometry to ensure that no changes had occurred and ES8 was validated by DNA fingerprinting at DSMZ (Braunschweig, Germany).

### Human NK Cell Expansion

Peripheral blood samples were obtained from discarded byproducts of platelet collections from healthy adult donors. Mononuclear cells were purified by centrifugation on an Accu-Prep density step (Accurate, Westbury, NY) and washed twice in RPMI-1640. To expand CD56+ CD3− NK cells, peripheral blood mononuclear cells and the genetically modified K562-mb15-41BBL cell line were co-cultured, as previously described in Fujisaki et al., Cancer Res, 69(9):4010-4017 (2009); Imai et al., Blood, 106:376-383 (2005)). Briefly, peripheral blood mononuclear cells were cultured with 100 Gy-irradiated K562-mb15-41BBL cell at 1.5:1 ratio in SCGM (CellGenix, Freiburg, Germany) containing 10% FBS, antibiotics and 10 IU/mL of recombinant human interleukin-2 (IL-2; Roche, Mannheim, Germany) in 6-well tissue culture plates. Tissue culture medium was partly exchanged every 2 days. After 7 days of co-culture, residual T cells were removed with Dynabeads CD3 (Invitrogen), resulting in cell population containing >95% CD56+ CD3− NK cells.

### Plasmids, Virus Production and Gene Transduction

The pMSCV-IRES-GFP, pEQ-PAM3(-E), and pRDF were obtained from the St. Jude Vector Development and Production Shared Resource. Interleukin-15 (IL-15) with a long signal peptide was sub-cloned by polymerase chain reaction (PCR) from a human spleen cDNA library (from Dr G. Neale, St Jude Children's Research Hospital) used as a template. The cDNA encoding the signal peptide of CD8α, the mature peptide of IL-15 and the transmembrane domain of CD8α were assembled by the splicing by overlapping extension by PCR (SOE-PCR) to encode a membrane-bound form of IL-15 (“mbIL15”); a wild-type form of IL-15 (not linked to CD8α transmembrane domain; “wtIL15”) was also tested prepared. The resulting expression cassettes were sub-cloned into EcoRI and XhoI sites of murine stem-cell virus-internal ribosome entry site-green fluorescent protein (MSCV-IRES-GFP).

To generate RD144-pseudotyped retrovirus, 3.0×10⁶ 293T cells were transfected using X-tremeGENE 9 DNA (Roche, Mannheim, Germany), maintained in 10-cm tissue culture dishes for 18h, with 3.5 μg of cDNA encoding mbIL15 constructs, 3.5 μg of pEQ-PAM3(-E), and 3 μg of pRDF. After replacing the medium with RPMI-1640 with 10% FBS and antibiotics at 24 hours, the conditioned medium containing retrovirus was harvested at 36-96 hours and added to polypropylene tubes coated with RetroNectin (Takara, Otsu, Japan), which were centrifugated at 1400 g for 10 min and incubated at 37° C. and 5% CO₂ for 4 hours. After additional centrifugation, and removal of the supernatant, expanded NK cells (0.5-1×10⁶) were added to the tubes and left in at 37° C. for 12 hours; these steps were repeated up to 6 times over 2-3 days. Cells were then maintained in RPMI-1640 with FBS, antibiotics and 100 IU/ml of IL-2. Transduced cells were assayed 3-29 days after transduction.

Surface expression of mbIL-15 was analyzed by flow cytometry using an anti-human IL-15 antibody (R&D, Minneapolis, MN) and phycoerythrin conjugated goat anti-mouse IgG1 (Southern Biotech, Birmingham, AL). Antibody staining was detected with a Fortessa flow cytometer (Becton Dickinson). Levels of IL-15 in culture supernatants were measured with the Quantikine Immunoassay (R&D).

### Functional Analysis of NK Cells In Vitro

To estimate NK cell survival and growth in vitro, transduced NK cells (1×10⁶ cells/mL) were resuspended in RPMI-1640 with 10% FBS and antibiotics, placed into the wells of either a 24- or a 96-well plate (Costar, Corning, NY) and cultured without or with IL-2 (10-100 IU/ml). Numbers of viable GFP+ cells were determined with an Accuri C6 flow cytometer (Becton Dickinson), after staining with propidium iodide. In some experiments, cells were incubated for 10 minutes with a neutralizing anti-IL-15 antibody (R&D) or an isotype-matched non-reactive antibody before culture.

NK cell immunophenotyping was performed using the antibodies listed in the Table, visualized with a Fortessa flow cytometer and analyzed by Diva (Becton Dickinson) and FlowJo (TreeStar, Ashland, OR) software. For phosphoprotein analysis, mock- and mbIL15-transduced NK cells (1×10⁷) were cultured without IL-2 for 48 hours. Cell lysates were prepared using a lysis buffer containing 20 mM 3-(N-morpholino) propanesulfonic acid, 2 mM EGTA, 5 mM EDTA, 30 mM sodium fluoride, 60 mM β-glycerophosphate, 20 mM sodium pyrophosphate, 1 mM sodium orthovanadate, 1% Triton X-100, Complete Mini protease inhibitor cocktail (Roche, Mannheim, Germany) and 1 mM dithiothreitol. After sonication, lysates were frozen at −80° C. and shipped in dry ice to Kinexus (Vancouver, CA) for Kinex Antibody Microarray analysis.

For cytotoxicity assays, luciferase-labeled target cells and NK cells (cultured without IL-2 for 48 hours) were plated in 96-well, flat-bottomed black Viewplates (Corning) at various effector: target (E: T) ratios and cultured for 4 or 24 hours. Adherent cell lines were incubated at 37° C. and 5% CO₂ for 4 hours before adding NK cells to allow for cell attachment. For antibody-dependent cell cytotoxicity assays, Rituximab (Rituxan, Roche; Mannheim, Germany),

Trastuzumab (Herceptin, Roche) or purified human IgG (R&D Systems, Minneapolis, MN) were added (all at 1 g/mL) before NK cells. At the end of the cultures, an equal volume of Bright-Glo luciferase reagent (Promega, Madison, WI) was then added to each test well, and after 5 minutes, luminescence was measured using a plate reader and analyzed with Gen5 2.00 software (both from BioTek, Tucson, AZ). In each plate, target cell viability was calculated using the luminescent signal from wells containing target cells only. All experiments were done in triplicate.

To measure release of lytic granules, NK cells (cultured for 48 hours without IL-2) were cocultured with K562, U937 cells, or 721.221 cells and their Cw6-expressing variant for 4 hours. We added PE- or PE-Cy7-conjugated anti-CD107a antibody (BD Biosciences) at the beginning of the cultures and GolgiStop (0.15 μL; BD Biosciences) 1 hour later. Percentage of CD107a+ NK cells was determined by flow cytometry.

### Expansion and Cytotoxicity of NK Cells in Immunodeficient Mice

To test NK cell expansion in vivo, human NK cells transduced with mbIL15 or mock-transduced (6-9×106 cells per mouse) were injected in the tail vein of NOD.Cg-Prkde^{scid} IL2rg^{tm1Wjl}/SzJ (NOD/scid IL2RGnull) mice (Jackson Laboratories, Bar Harbor, ME). In some mice, we injected 20000 IU of IL-2 intraperitoneally (i.p.) 3 times per week. On day 7 and 11, blood cells were counted with a cell counter (Beckman Coulter); human and mouse CD45+ cells were enumerated by flow cytometry after treating cells with red cell blood lysis solution (Invitrogen) and staining them with an allophycocyanin-conjugated mouse-anti-human CD45 and a phycoerythrin-conjugated rat anti-mouse CD45 antibodies (both from BD Biosciences). After euthanasia, human NK cells in bone marrow, liver, spleen, kidney, lung, and brain were enumerated as above. All animal experiments were performed in accordance a protocol approved by National University of Singapore Institutional Animal Care and Use Committee.

To test tumor cell killing in mice, we prepared two xenograft models. In the first, U937 cells expressing luciferase were injected i.p. in NOD.Cg-Prkdc^{scid} IL2rg^{tm1Wjl}/SzJ (NOD/scid IL2RGnull) mice (1×10⁴ cells per mouse). Three days later, NK cells transduced with the MSCV vector containing either GFP alone or mbIL15 were injected i.p. (1×10⁷ cells per mouse); NK cell injection was repeated on day 7. As a control, a group of mice received tissue culture medium instead of NK cells. In the second model, mice were engrafted with ES8 cells (i.p.; 1×10⁵ cells per mouse), followed by 1 NK cell injection on day 3 as above. Tumor engraftment and progression was evaluated using a Xenogen IVIS-200 system (Caliper Life Sciences, Hopkinton, MA), with imaging beginning 5 minutes after i.p. injection of an aqueous solution of D-luciferin potassium salt (3 mg/mouse). Photons emitted from luciferase-expression cells were quantified using the Living Image 4.3.1 software program.

### Results

### Design of IL-15 Constructs and Expression in NK Cells

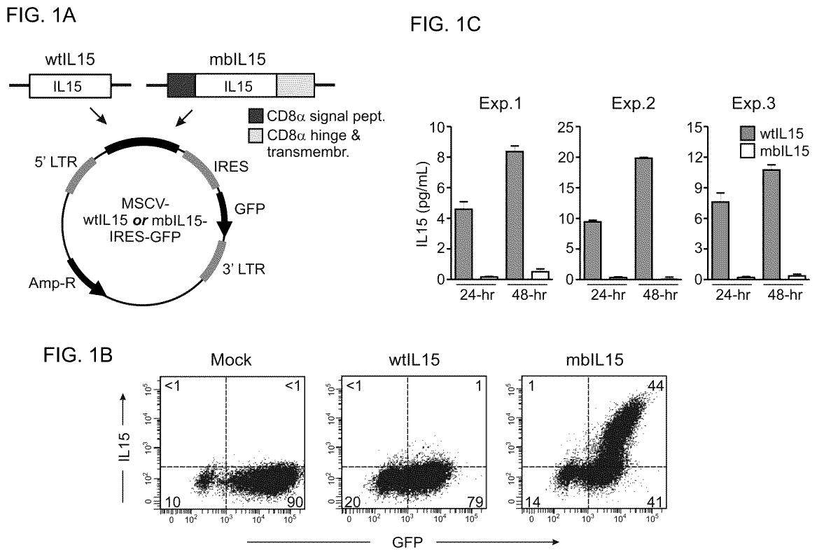

As described herein, two forms of the IL15 gene were expressed in human NK cells: a membrane-bound form, resulting from a construct in which the human IL15 gene was linked to the gene encoding the transmembrane domain of CD8α (“mbIL15”), and a wild-type unmodified form (“wtIL15”). Both constructs were inserted in an MCSV retroviral vector containing GFP (FIG. 1A), which was used to transduce proliferating NK cells obtained after culturing peripheral blood mononucleated cells with the stimulatory cell line K562-mb15-41BBL.28 At the end of the cultures, before retroviral transduction, residual T-cells were depleted with anti-CD3 immunomagnetic beads resulting in >95% pure CD56+CD3-cells. Median GFP expression was 71% (23%-97%, n=60) with the construct containing mbIL15, and 69% (range, 20%-91%, n=25) with that containing wtIL15. NK cells from the same donors also transduced with a vector containing only GFP had a median GFP expression of 84% (53%-98%, n=60) (FIG. 1B).

After transduction with mbIL15, IL-15 was expressed on the NK cell membrane: 40%-63% (median, 52; n=7) of GFP+NK cells had IL-15 as detected by an anti-IL15 antibody (FIG. 1B). By contrast, no IL-15 was detectable in cells transduced with wtIL15 (n=4) or mock transduced NK cells (n=7). Production of soluble IL-15 by the transduced NK cells was determined by testing supernatants collected after 24 and 48 hours of culture. A shown in FIG. 1C, cells expressing wtIL15 secreted substantial amounts of IL-15 whereas this was minimal in mbIL15-NK cells and undetectable in mock-transduced NK cells.

Nk Cells Expressing IL-15 have Autonomous Survival and Expansion Capacity

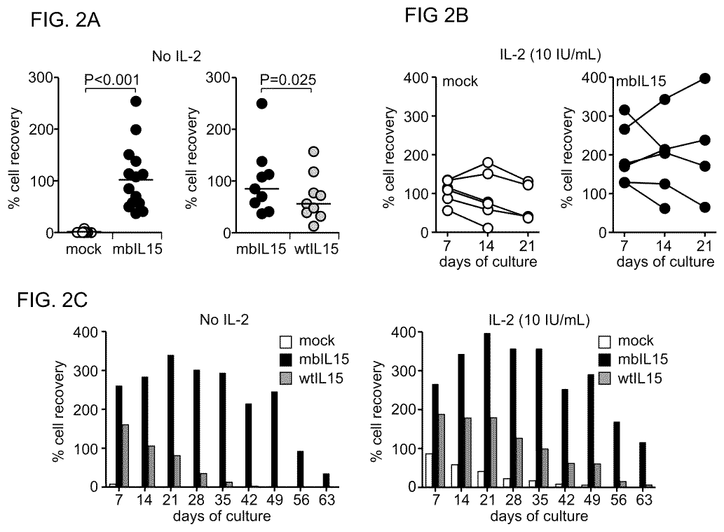

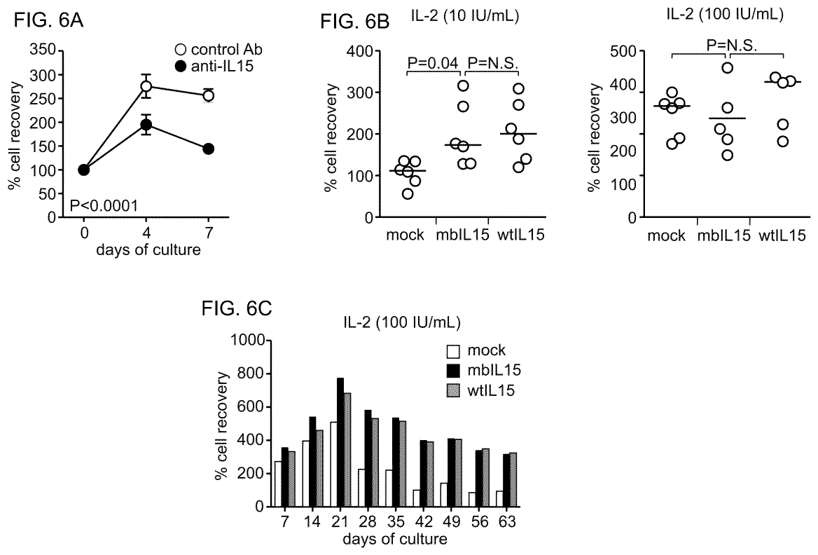

To determine whether expression of IL-15 could replace exogenous IL-2 in maintaining NK cell survival, NK cells from 15 donors were transduced with the mbIL15 construct and cultured in the absence of IL-2; cell numbers after culture were then compared to those in parallel cultures with mock-transduced NK cells. As shown in FIG. 2A, expression of mbIL-15 dramatically increased NK cell survival: after 7 days of culture, median cell recovery was 85% while virtually no viable mock-transduced NK cell was detectable (<1%; P<0.0001 by paired t test). The effect of mbIL15 significantly decreased if an anti-IL-15 neutralizing antibody was added to the cultures (FIGS. 6A-6B). In 9 of the 15 donors, recovery of mbIL15 NK cells was also compared to that of NK cells expressing wtIL15: it was significantly higher with the former (median, 85% versus 56%, P=0.026; FIG. 2A).

In parallel experiments, the supportive effects of IL15 expression in the presence of exogenous IL-2 were determined. When cultures contained 10 IU/mL of IL-2, 7-day recovery of NK cells expressing either mbIL15 or wtIL15 remained significantly higher than that of mock-transduced cells; under these conditions, no significant differences were noted between the 2 forms of IL15 (FIGS. 6A-6B). Only when exogenous IL-2 was present at a high concentration (100 IU/mL), 7-day recovery of mock-transduced NK cells matched that of NK cells transduced with IL15 (FIG. 6A).

In experiments with expanded NK cells from 6 of the 9 donors, the capacity of mbIL15 to support NK cell survival beyond 7 days with low dose IL-2 (10 IU/mL) was determined. On day 14, mbIL15 NK cell numbers were maintained or increased in 4 of the 6 cultures; in 2 of these cells had further expanded by day 21. Only 2 of the 6 cultures with mock-transduced NK cells from the same donors had maintained cell numbers on day 14 and 21, and no cell growth was observed; median cell recovery on day 21 was 205% for mbIL15 NK cells and 80% for mock-transduced NK cells. Thus, even in the presence of low dose IL-2, expression mbIL15 conferred a considerable survival and growth advantage.

In cultures of NK cells from one donor, a particularly high cell recovery was observed on day 7 when IL15 was expressed (261% with mbIL15 and 161% with wtIL15 in the absence of IL-2; 266% and 188% with 10 IU/mL IL-2). These cultures were monitored for 2 months and remarkable improvements in cell expansion and survival brought about by the expression of mbIL15 were observed (FIG. 2C). Even in the absence of IL-2, mbIL-15 NK cells continued to survive until day 21 and they were still detectable 75 days after initiation of the culture, while mock-transduced cells had become undetectable on day 7 and wtIL15-transduced NK cells on day 42. In the presence of IL-2 at low concentration (10 IU/mL), the number of mbIL15-expressing NK cells was identical to that originally seeded 2 months after initiation of the cultures, while viable mock-transduced and wtIL15-transduced NK cells had declined much earlier. As shown in FIG. 6B, when IL-2 was added to the culture at a high dose (100 IU/mL), NK cells transduced with either mbIL155 or wtIL15 had a similar persistence profile, both cell types surviving longer than mock-transduced NK cells even under these conditions.

Expansion and Homing of mbIL15 NK Cells In Vivo

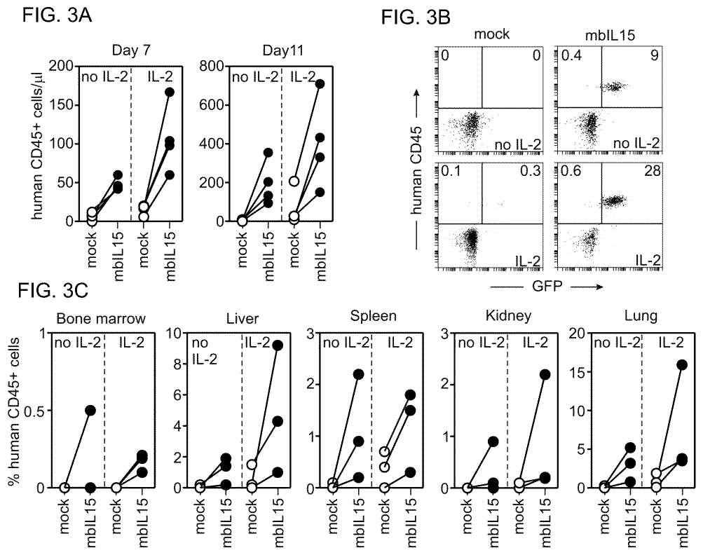

The experiments performed in vitro indicated that IL15 expression improved survival and expansion of NK cells and that mbIL15 produced overall better stimulation. Whether mbIL15 expression would sustain expansion of human NK cells in NOD/scid IL2RGnull mice was next determined. Activated NK cells from 4 donors were transduced with mbIL15 (52%-74% GFP-positive) and injected into 4 mice (one mouse per donor); 4 control mice were injected with mock-transduced NK cells from the same donors. NK cells expressing mbIL15 expanded much more than mock-transduced NK cells: 7 days after injection, median number of mbIL15 NK cells/μl of blood was 44.5 (range, 42-60) versus 6.5 (0-12) with mock-transduced NK cells (P=0.004) (FIG. 3A). Parallel experiments were performed with the same cells, this time also administering 20,000 IU human IL-2 i.p. every 2 days (FIG. 3A). Under these conditions, mbIL15 NK cells expanded even more (median NK cells/μl, 101; range, 60-167), while mock-transduced cells remained low (median, 18; range, 6-20; P=0.021).

On day 11 after injection, mbIL15 NK cells comprised 168.5 cells/μl (range, 94-355) of peripheral blood mononucleated cells in the absence of IL-2 and 382 cells/μl (151-710) when IL-2 was also administered (FIG. 3A, B). By contrast, in mice injected with mock-transduced NK cells, human CD45 cells were virtually undetectable without IL-2, and present at low levels when IL-2 was also injected (median, 27; range 9-207; P=0.026). Human CD45± cells also expressed CD56 and lack CD3 (not shown). Of note, the proportion of GFP± had increased from 66.5%±9.9% before injection to 93.8%±4.4% on day 7 and 94.8%±3.4% on day 11 (P<0.01 for both comparisons).

After euthanasia on day 11, 3 of the 4 mice were examined for the presence of human CD45+ cells in various tissues. If mbIL15 was expressed, considerable numbers of human NK cells were detected in bone marrow, liver, spleen, kidney and lung; in all tissues, numbers were markedly higher than those seen with mock-transduced cells (FIG. 3C): mean (±SD) percentage of CD45+ cells expressing mbIL15 was 1.2%±1.5% with no IL-2 and 3.0%±4.3% with IL-2, as compared to 0.04%±0.09% and 0.4%±0.6% with mock-transduced cells (P<0.001 and P=0.002, respectively). The only exception was brain where neither mbIL15-nor mock-transduced NK cells could be detected.

Mechanisms of mbIL15 Stimulation

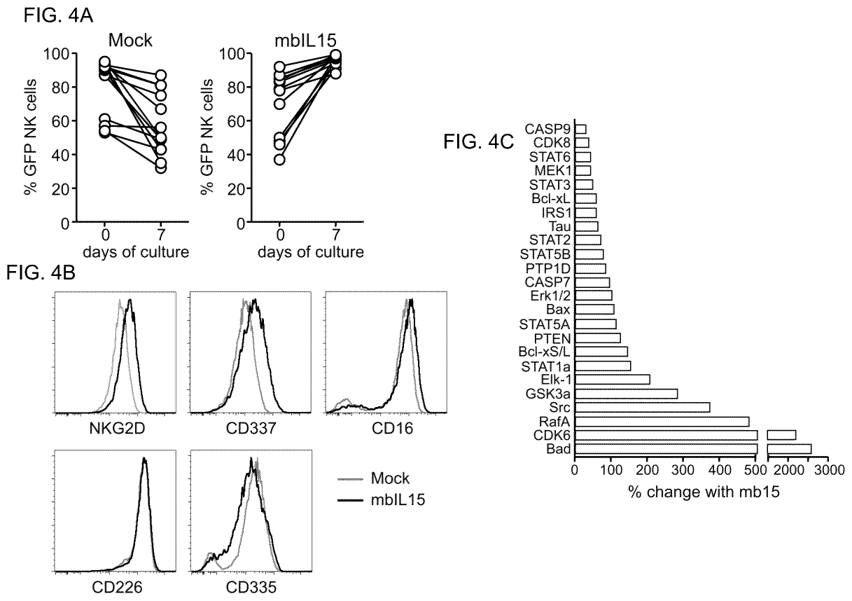

To determine whether mbIL15 predominantly stimulated cells in trans (IL-15 presented on one NK cell stimulating a neighboring cell (a mechanism reported to occur physiologically)) or cis (by direct binding of mbIL15 to receptors expressed in the same cell), the proportions of GFP+ and GFP-NK cells in the cultures were evaluated after 7 days of culture. If the trans mechanism was predominant, the ratio between GFP+ and GFP-NK cells should remain unaltered during culture; if cis was predominant, the proportion of GFP+ cells should increase. FIG. 4A shows the results of such analysis: the percentage of GFP+ cells among NK cells examined after 7 days of culture without IL-2 consistently increased if mbIL15 was expressed whereas it did not in cultures with mock-transduced cells: GFP+ cells constituted 95.9%±3.3% of the total cell population versus 57.5%±18.6% on day 7 (P<0.0001), as compared to 71.2%±19.0% versus 80.5%±17.1% on day 0. Thus, the predominant mechanism of stimulation by mbIL-15 expressed in NK cells is autocrine.

Cells expressing mbIL15 essentially retained the immunophenotype of activated NK cells. However, when examined 2 days after IL-2 withdrawal compared to mock-transduced NK cells, mbIL15 NK cells expressed moderately higher levels of the activation receptors NKG2D, NKp44 (CD336) and NKp30 (CD337) as well as of CD16 and CD56, while expression of NKp46 (CD335) decreased and that of other molecules, such as DNAM-1 (CD226), remained unchanged (FIG. 4B; the Table). The signal transduction pathways activated by the expression of mbIL15 were also determined. As shown in FIG. 4C, in comparison to mock-transduced NK cells, mbIL-15 NK cells had several highly phosphorylated molecules. These included molecules known to be phosphorylated in response to IL-15 signaling, such as the transcription factors STAT1, STAT3 and STAT5, the kinases src, Erk1/2 and Mek1. Notably, a marked phosphorylation of Bad, as well as phosphorylation of Caspase 7 and 9, collectively indicative of an anti-apoptotic effect, were observed. Other highly phosphorylated molecules in mbIL15 NK cells whose role in IL-15 signaling is unclear included CDK6 and RafA.

Effects of mbIL-15 on NK Cell Anti-Tumor Cytotoxicity In Vitro and In Vivo

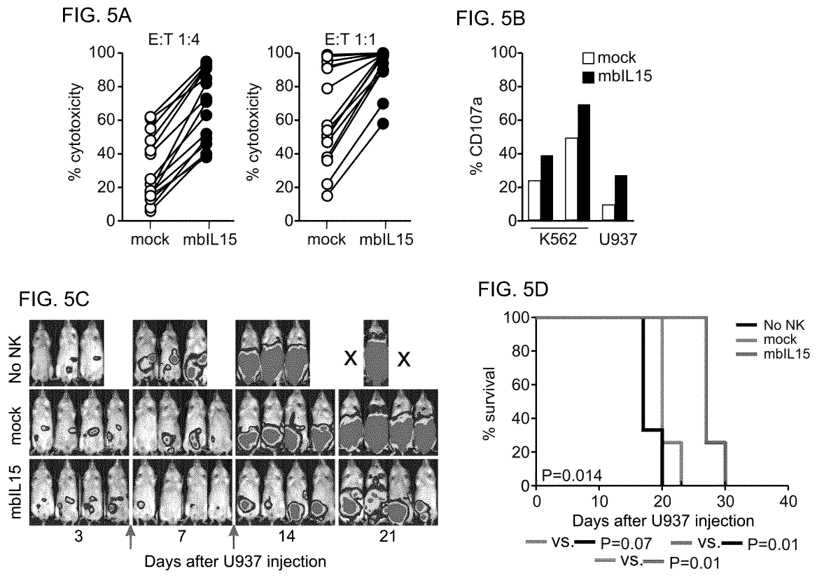

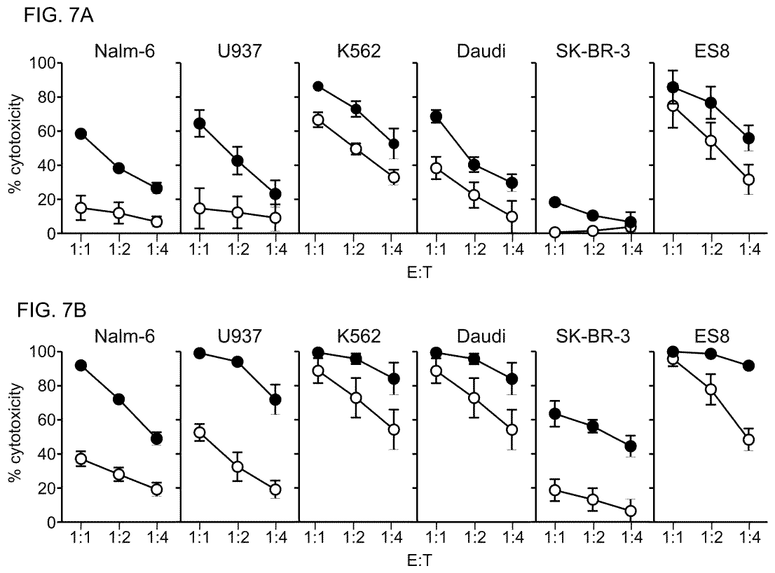

The improvements in NK cell survival and proliferation brought about by expression of mbIL15 indicated that NK-mediated killing of tumor cells would likely also increase. This notion was first tested by comparing tumor cell cytotoxicity exerted by mbIL15-NK cells to that of mock transduced NK cells from the same donors. Experiments with NK cells from 9 donors targeting the leukemia cell lines Nalm-6 (B-lineage acute lymphoblastic leukemia), U937 and K562 (acute myeloid leukemia), as well as Daudi (B-cell lymphoma), SKBR3 (breast carcinoma) and ES8 (Ewing sarcoma) at different E: T ratios and co-culture durations, for a total of 90 experiments, were performed. FIG. 5A shows results of 24-hour assays: median cytotoxicity was 22% with mock-transduced NK cells at 1:4 E: T and 54% at 1:1 E: T; with mbIL15 NK cells, it was 71% and 99%, respectively (P<0.0001). Results with individual cell lines are shown in FIGS. 7A-7B. Although the increased cytotoxicity might be related to the increase survival of NL cells in culture, an increased release of lytic granules by mbIL15-NK cells, as revealed by CD107a staining after culture with either K562 or U937 cells, was also observed (P=0.0067; FIG. 5B).

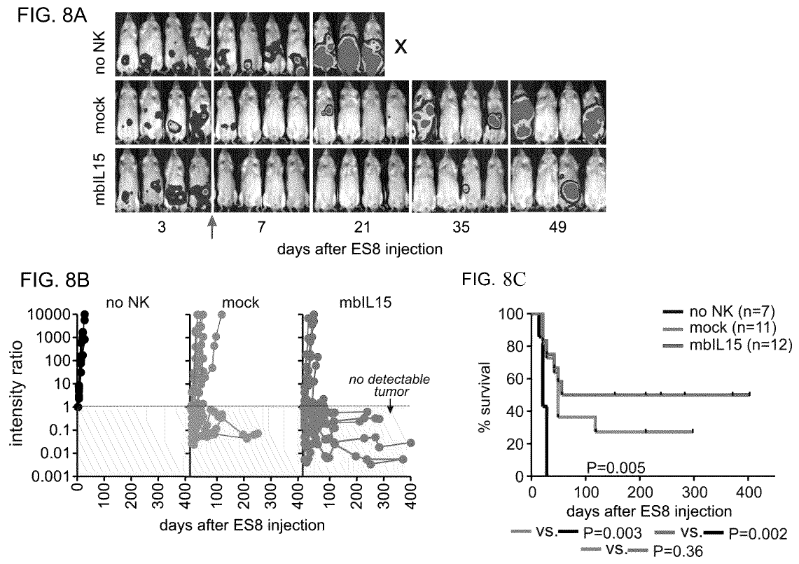

The gains in vitro cytotoxicity associated with expression of mbIL15 were reflected in experiments with NOD/scid IL2RGnull mice engrafted with human tumor cells. In one set of experiments, mice were injected with the human acute myeloid leukemia (AML) cell line U937 and then treated with either mbIL15- or mock-transduced NK cells. As shown in FIGS. 5C and 5D, mice receiving mbIL15-transduced NK cells had a slower tumor growth and a significantly longer survival than untreated mice and those treated with mock-transduced NK cells (P=0.014, log rank test for trend). The cells were also tested in a second xenograft model in which NOD/scid IL2RGnull mice were injected with the Ewing sarcoma cell line ES8, which has a much slower growth rate, and the mice were treated with one injection of NK cells. As shown in FIGS. 8A-8C, the outcome of mice treated with mbIL15 NK cells (n=12) was superior to that of mock-transduced NK cells (n=11) and of untreated mice (n=7): median survival was 162, 49 and 21 days, respectively (P=0.005).

### Discussion

Among the factors that determine the success of NK cell-based therapy of cancer, perhaps the most fundamental one is that NK cells persist in sufficient numbers to achieve an E: T ratio likely to produce tumor cytoreduction. Demonstrated herein is that expression of a membrane-bound form of IL-15 in human NK cells supported their autonomous expansion and extended survival in the absence of IL-2. NK cells expressing mbIL15 could be maintained in vitro for up to 2 months without exogenous IL-2. NK cells expressing mbIL15 could expand in immunodeficient mice and infiltrated multiple tissues where they could be found in much larger numbers than mock-transduced cells. Expansion of mbIL-15 NK cells was further increased by a low concentration of IL-2 both in vitro and in vivo. Expression of mbIL15 did not impair the cytotoxic capacity of NK cells. In fact, in xenograft models, mbIL15 NK cells exerted anticancer activity which was more powerful than that of mock-transduced cells, indicating that this approach might improve the antitumor capacity of NK cell infusions while averting the side effects of IL-2 administration.

The findings herein show that ectopic expression of IL-15 in human NK cells caused a stronger survival-promoting effect when IL-15 was presented in a membrane-bound form than in a secreted form. Notably, however, mbIL15 expressed in NK cells preferentially stimulates in cis rather than in the trans when IL-15 is presented by other cells. That is, mbIL15 appears to preferentially engage IL-15 receptors on the same cells, resulting in autocrine stimulation. This mechanism explains the IL-15 expression pattern that was consistently observed when mbIL15-transduced NK cells were labeled with an anti-IL-15 antibody, showing a substantial proportion of cells with strong GFP expression but ostensibly lacking IL-15 (FIG. 1B). It is hypothesized that in these cells IL-15 is expressed but not accessible to antibody because it is bound to its receptor and/or internalized. The capacity of mbIL15 to promote NK cell viability likely explains the increased cytotoxicity exerted by these cells, particularly in 24-hour in vitro assays and in vivo. However, the superiority of mbIL15-NK cells was also clear in short-term (4-hour) assays and these cells also released more lytic granules according to the CD107a test. Therefore, expression of mbIL15 is likely to increase NK cell cytotoxicity by other means, possibly by enhancing their activation status.

Clinical administration of NK cells typically relies on IL-2 to support their survival and expansion in vivo. The multiple side effects related to IL-2 administration, however, are potentially serious and often render administration of this cytokine poorly tolerated. Stopping IL-2 administration or reducing its dose may results in decreased NK cell expansion and inefficient anti-tumor effect, which may be further inhibited by the stimulation of regulatory T cells. To this end, replacing IL-2 with IL-15 is potentially attractive but the clinical formulation of IL-15 is still being tested. Although it was overall well tolerated when administered to rhesus macaques, adverse effects were observed in some animals, including diarrhea, emesis, weight loss, transient neutropenia, increase in transaminases and hyponatremia. In addition to T and NK cell expansion, expansion of regulatory T cells has been observed. Contrary to NK cells transduced with wtIL15, those transduced with mbIL15 released exceedingly small amount of IL-15 in the supernatant. Thus, any potential side effect that may be caused by the interaction of IL-15 with cells other than NK cells should be minimized by this approach. Of note, prolonged exposure of murine large granular lymphocytes to IL-15 leads to their leukemic growth. This poses a potential safety concern for IL-15 administration in patients and also for the use of NK cells expressing IL-15, particularly if such cells were administered to patients at a low risk of relapse. In the experiments described herein, however, NK cells expressing mbIL15 generally survived for much shorter periods than the one year or more reported for T cell clones expressing soluble IL-15. Moreover, persistent NK expansion was not observed in immunodeficient mice, with a follow-up exceeding 9 months.

There is considerable clinical evidence supporting the anti-cancer potential of NK cells. NK cells also play a critical role in mediating antibody-dependent cell cytotoxicity in patients treated with monoclonal antibodies. Thus, infusion of NK cells is likely beneficial in multiple settings. Expansion of human NK cells in large numbers ex vivo is feasible; robust large-scale methods for this purpose have been established and are being used in clinical trials. Genetic modification of NK cells by retroviral transduction or electroporation is also possible. Therefore, the translation of the approach described herein into clinical-grade conditions is realistic and it is warranted by the superior expansion and cytotoxicity of mbIL15-NK cells.

TABLE
Surface marker expression in mock-
and mbIL15 transduced NK cells¹

| | Mock | | mb15 | |
|---|---|---|---|---|
| Marker | %² | MFI | % | MFI |
| CD56 | 100 | 44190 | 100 | 56721³ |
| CD16 | 88 | 7789 | 92 | 10784 |
| CD69 | 90 | 3057 | 91 | 4481 |
| CD25 (IL2Rα) | 56 | 631 | 75 | 795 |
| CD122 (IL2Rβ/IL15Rβ) | 100 | 4833 | 99 | 3216 |
| CD132 (IL2Rγ) | 89 | 943 | 97 | 1263 |
| NKG2D | 99 | 2846 | 100 | 4953 |
| CD335 (NKp46) | 89 | 2613 | 89 | 2236 |
| CD336 (NKp44) | 84 | 9455 | 83 | 11530 |
| CD337 (NKp30) | 91 | 1286 | 95 | 2678 |
| CD226 (DNAM-1) | 99 | 16440 | 99 | 16905 |
| CD158ah (KIR2DL1, KIR2DS1 | 23 | 6747 | 24 | 11793 |
| CD158b | 49 | 42515 | 47 | 51247 |
| CD158e | 22 | 4225 | 22 | 4946 |
| CD159a | 68 | 18133 | 73 | 21106 |

¹Cell markers were analyzed after 48 hours of culture in the absence of IL-2. Antibodies were from BD Biosciences (CD56 PE, CD16 PE-Cy7, CD69 PE, CD25 PE-Cy7, CD122 BV421, CD158b PE), Beckman Coulter (CD335 PE, CD336 PE, CD337 PE, CD158ah PE, CD159a PE), Miltenyi Biotech (CD226 PE, CD158e APC), R&D Systems (NKG2D PE), Biolegend (CD132 APC).
²Percentages refer to GFP+ cells expressing the marker.
³Overexpressed markers are highlighted in bold font.
MFI, mean fluorescence intensity

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A natural killer (NK) cell that expresses all or a functional portion of interleukin-15 (IL-15), wherein IL-15 is expressed as a membrane-bound polypeptide.

2. The NK cell of claim 1, wherein the all or a functional portion of IL-15 is full-length IL-15.

3. The NK cell of claim 1, wherein the all or a functional portion of IL-15 is fused to all or a portion of a transmembrane protein.

4. The NK cell of claim 3, wherein the transmembrane protein comprises a CD8 transmembrane domain.

5. A composition comprising the NK cell of claim 1.

6. A method of producing a natural killer (NK) cell that expresses all or a functional portion of interleukin-15 (IL-15) comprising:
a) introducing, into a NK cell, a nucleic acid encoding all or a functional portion of IL-15, wherein IL-15 is expressed as a membrane-bound polypeptide; and
b) maintaining the NK cell under conditions in which the all or a functional portion of the IL-15 is expressed.

7. The method of claim 6, wherein the nucleic acid introduced into the NK cell encodes a signal peptide of CD8a, the all or a functional portion of IL-15, and a transmembrane protein comprising a CD8 transmembrane domain.

8. The method of claim 6, wherein the nucleic acid is introduced into the NK cell with a viral vector.

9. A method of treating a cancer in an individual in need thereof, the method comprising administering to an individual having a cancer a natural killer (NK) cell that expresses all or a functional portion of interleukin-15 (IL-15), wherein IL-15 is expressed as a membrane-bound polypeptide.

10. The method of claim 9, wherein the cancer is a leukemia or a lymphoma.

11. The method of claim 9, wherein the method further comprises administering IL-2 to the individual.

12. A method of enhancing the expansion and/or survival of natural killer (NK) cells comprising:
a) introducing, into NK cells, a nucleic acid encoding all or a functional portion of interleukin-15 (IL-15); and
b) maintaining the NK cells under conditions in which the all or a functional portion of the IL-15 is expressed and the NK cells proliferate,
wherein the method increases the in vivo expansion and/or survival of the NK cells and/or the method increases the in vitro or ex vivo expansion and/or survival of the NK cells.
